SEQUENCE LISTING (2 sequences)

SEQ ID NO: 1 - DNA, 3000 bp, Homo sapiens
  CDS (66)..(2840): CDS
  misc_feature (126)..(419): first Immunoglobulin-like domain
  misc_feature (2556)..(2645): transmembrane region
ccgtcctctagcccagctcctcggctcgcgctctcctcgcctcctgtgctttccccgccgcggcgatgccagggccttcgccagggctgcgccgggcgctactcggcctctgggctgctctgggcctggggctcttcggcctctcagcggtctcgcaggagcccttctgggcggacctgcagcctcgcgtggcgttcgtggagcgcgggggctcgctgtggctgaattgcagcaccaactgccctcggccggagcgcggtggcctggagacctcgctgcgccgaaacgggacccagaggggtttgcgttggttggcgcggcagctggtggacattcgcgagccggagactcagcccgtctgcttcttccgctgcgcgcggcgcacactacaggcgcgtgggctcattcgcactttccagcgaccagatcgcgtagagctgatgccgctgcctccctggcagccggtgggcgagaacttcaccctgagctgtagggtccccggcgccgggccccgtgcgagcctcacgctgaccctgctgcggggcgcccaggagctgatccgccgcagcttcgccggtgaaccaccccgagcgcggggcgcggtgctcacagccacggtactggctcggagggaggaccatggagccaatttctcgtgtcgcgccgagctggacctgcggccgcacggactgggactgtttgaaaacagctcggcccccagagagctccgaaccttctccctgtctccggatgccccgcgcctcgctgctccccggctcttggaagttggctcggaaaggcccgtgagctgcactctggacggactgtttccagcctcagaggccagggtctacctcgcactgggggaccagaatctgagtcctgatgtcaccctcgaaggggacgcattcgtggccactgccacagccacagctagcgcagagcaggagggtgccaggcagctgatctgcaacgtcaccctggggggcgaaaaccgggagacccgggagaacgtgaccatctacagcttcccggcaccactcctgaccctgagcgaacccagcgtctccgaggggcagatggtgacagtaacctgcgcagctgggacccaagctctggtcacactggagggagttccagccgcggtcccggggcagcccgcccagcttcagctaaatgccaccgagaacgacgacagacgcagcttcttctgcgacgccaccctcgatgtggacggggagaccctgatcaagaacaggagcgcagagcttcgtgtcctatacgctccccggctagacgattcggactgccccaggagttggacgtggcccgagggcccagagcagacgctgcgctgcgaggcccgcgggaacccagaaccctcagtgcactgtgcgcgctccgacggcggggccgtgctggctctgggcctgctgggtccagtcactcgggcgctctcaggcacttaccgctgcaaggcggccaatgatcaaggcgaggcggtcaaggacgtaacgctaacggtggagtacgcaccagcgctggacagcgtgggctgcccagaacgcattacttggctggagggaacagaagcctcgctgagctgtgtggcgcacggggtaccgccgcctgatgtgatctgcgtgcgctctggagaactcggggccgtcatcgaggggctgttgcgtgtggcccgggagcatgcgggcacttaccgctgcgaagccaccaaccctcggggctctgcggccaaaaatgtggccgtcacggtggaatatggccccaggtttgaggagccgagctgccccagcaattggacatgggtggaaggatctgggcgcctgttttcctgtgaggtcgatgggaagccacagccaagcgtgaagtgcgtgggctccgggggcgccactgagggggtgctgctgccgctggcacccccagaccctagtcccagagctcccagaatccctagagtcctggcacccggtatctacgtctgcaacgccaccaaccgccacggctccgtggccaaaacagtcgtcgtgagcgcggagtcgccaccggagatggatgaatctacctgcccaagtcaccagacgtggctggaaggggctgaggcttccgcgctggcctgcgccgcccggggtcgcccttccccaggagtgcgctgctctcgggaaggcatcccatggcctgagcagcagcgcgtgtcccgagaggacgcgggcacttaccactgtgtggccaccaatgcgcatggcacggactcccggaccgtcactgtgggcgtggaataccggccagtggtggccgaacttgctgcctcgccccctggaggcgtgcgcccaggaggaaacttcacgttgacctgccgcgcggaggcctggcctccagcccagatcagctggcgcgcgcccccgggggccctcaacatcggcctgtcgagcaacaacagcacactgagcgtggcaggcgccatgggaagccacggcggcgagtacgagtgcgcacgcaccaacgcgcacgggcgccacgcgcggcgcatcacggtgcgcgtggccggtccgtggctatgggtcgccgtgggcggcgcggcggggggcgcggcgctgctggccgcgggggccggcctggccttctacgtgcagtccaccgcctgcaagaagggcgagtacaacgtgcaggaggccgagagctcaggcgaggccgtgtgtctcaacggagcgggcggcggcgctggcggggcggcaggcgcggagggcggacccgaggcggcggggggcgcggccgagtcgccggcggagggcgaggtcttcgccatacagctgacatcggcgtgagccgctcccctctccccgcgggccgggggacgccccccagactcacacgggggcttatttattgctttatttatttacttattcatttatttatgtattcaactccaagggcgtcacccccattttctacccatcccctcaataaagtttttataaagga

SEQ ID NO: 2 - protein, 924 aa, Homo sapiens
  misc_feature (21)..(118): first Immunoglobulin-like domain
  misc_feature (831)..(860): transmembrane region
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLICNVTLGGENRETRENVTIYSFPAPLLTLSEPSVSEGQMVTVTCAAGTQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGATEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPGALNIGLSSNNSTLSVAGAMGSHGGEYECARTNAHGRHARRITVRVAGPWLWVAVGGAAGGAALLAAGAGLAFYVQSTACKKGEYNVQEAESSGEAVCLNGAGGGAGGAAGAEGGPEAAGGAAESPAEGEVFAIQLTSA

(19) United States
(12) Patent Application Publication
(10) Pub. No.: US 2008/0057054 A1
(43) Pub. Date: Mar. 6, 2008

(54) Modulation of Phagocytosis in Neurons

(75) Inventors: Wim Annaert, Kontich (BE); Tim Raemaekers, Heverlee (BE)

Correspondence Address: TRASK BRITT, P.O. BOX 2550, SALT LAKE CITY, UT, 84110, US

(73) Assignees: Vlaams Interuniversitair Instituut Voor Biotechnol, Zwijnaarde (BE); K. U. Leuven Research and Development, Leuven (BE)

(21) Appl. No.: 11/662,528

(22) PCT Filed: Sep. 13, 2005
(86) PCT No.: PCT/EP05/54557

§ 371 Date: Jul. 2, 2007

(30) Foreign Application Priority Data
Sep. 13, 2004 (EP) 04077519.9

(51) Int. Cl. A61K 31/70; A61K 39/395; A61P 43/00; C12N 5/06

(52) U.S. Cl. 424/130.1; 435/375; 514/44

(57) ABSTRACT

The invention relates to the field of phagocytosis and more particularly to the field of neurons and neuron diseases. In particular the invention relates to the use of molecules capable of modulating the phagocytosis in neurons, more particularly to molecules modulating the activity of telencephalin.

### FIELD OF THE INVENTION

The invention relates to the field of phagocytosis and more particularly to the field of neurons and neuron diseases. In particular the invention relates to the use of molecules capable of modulating the phagocytosis in neurons, more particularly to molecules modulating the activity of telencephalin.

### BACKGROUND OF THE INVENTION

Phagocytosis is the process by which living cells ingest or ingulf other cells, tissue debris or foreign particles. In higher animals phagocytosis is chiefly a defensive reaction against infection and invasion of the body by foreign substances but the process is also needed to clean up tissue debris resulting from wound repair and apoptosis. This ingestion, which is performed most efficiently by migrating, bone marrow-derived cells called ‘professional phagocytes’, is essential for successful host defense. For example the ingestion of a microorganism results when an invading microorganism is recognized by specific receptors on the phagocyte surface and requires multiple, successive interactions between the phagocyte and the target. Each of these interactions results in a signal transduction event, which is confined to the membrane and cytoskeleton around the ligated receptor and which is required for successful phagocytosis. The particles commonly phagocytosed by leukocytes include bacteria, dead tissue cells, protozoa, various dust particles, pigments, and other minute foreign bodies such as microspheres and liposomes. In humans and in vertebrates generally, the most effective phagocytic cells are the macrophages (large phagocytic cells) and the granular leukocytes, or granulocytes (small phagocytic cells). The macrophages occur especially in the liver, spleen, and lymph nodes, in which their function is to free the blood and lymph of bacteria and other particles. Macrophages are also found in all tissues as wandering amoeboid cells, and the monocyte, a precursor of the macrophage, is found in the blood. The smaller granulocytes are white blood cells, mainly neutrophils, that are carried along by the circulating blood until they reach an area of infected tissue, where they pass through the blood vessel wall and lodge in that tissue. Phagocyting cells have cell surface receptors for the Fc domain of an IgG antibody. These receptors mediate phagocytosis and clearance of immune complexes. In the last decade it has become apparent that the over- or under-functioning of phagocytosis is at the basis of several human immune pathologies. For example macrophage Fc receptor function has been found to be decreased in patients with lupus erythematosis, Sjogren's syndrome and in end stage renal disease while an increase in phagocytosis has been found due to release of bacterial products, in rheumatoid arthritis and in autoimmune tissue destruction. This knowledge has been exploited in several ways wherein scientists have managed to modulate the phagocytosis pathway in either positive (US6,475,997) or negative modes (U.S. Pat. No. 6,638,764). In addition various carrier systems have emerged to deliver drugs specifically to macrophages (Ahsan F. et al (2002) J. Control Release 79:29). A region in the body which is not amenable for specific protein targeting is the human brain, and more particularly the neurons residing in the telencephalon. The telencephalon is an important region of the brain and includes the cerebral neocortex, paleocortex, the hypocampus, septum, striatum and the olfactory bulb. The telencephalon is also the brain region which is affected first in most neurodegenerative diseases.

In the present invention we have surprisingly found that the transmembrane receptor telencephalin is able to promote phagocytosis in neurons. Telencephalin (ICAM-5) is a cell adhesion molecule belonging to the immunoglobulin superfamily, whose expression is restricted to neurons residing in the telencephalon (Oka S. et al (1990) Neuroscience 35(1): 93, Yoshihara Y. et al (1994) Neuron 12(3):541). It is known in the art that the immunoreactivity of telencephalon is reduced in the brain of patients with Alzheimer's disease (Hino H. et al (1997) Brain Res. 753(2):353) and it is also known that soluble telencephalin is formed (by shedding) in pathological processes such as hypoxic-ischemic injury and in acute encephalitis but it has never been shown in the art that neurons are able to phagocytise neither has it been suggested that telencephalon could be involved in this process. The present invention can be exploited to modulate the process of phagocytosis in neurons. Since the telencephalon is the most important brain region affected in several neurodegenerative diseases it is desirable and now possible to design drugs (e.g. through the encapsulation in to microspheres or liposomes) with a specificity for neurons in the telencephalin. In addition the invention can be exploited in a reverse way since the inhibition of phagocytosis, by the use of for example an antibody against telencephalon, can be used to prevent the infection (through interference with phagocytosis) of pathogenic bacteria and viruses in the telencephalon.

### LEGENDS TO FIGURE

FIG. 1: 1.1 Nucleotide sequence of human telencephalin, 1.2 Amino acid sequence of human telencephalin. The shaded area represents the first immunoglobulin-like domain, the transmembrane region is boxed.

### AIMS AND DETAILED DESCRIPTION OF THE INVENTION

The central nervous system (CNS) is a very attractive target for new therapeutic strategies since many genes involved in neurological diseases are known and often only local low level gene expression or a low level of a therapeutic protein or therapeutic compound is required to interfere with the disease process. However, as the blood brain barrier on one hand prevents some therapeutic agents given systematically from exerting their activity in the CNS, it also provides an immune privileged environment. Neurosurgical technology (e.g. by means of pumps) meanwhile allows the access of nearly every single center of the CNS and provides the surgical tool for direct gene, protein or compound delivery via minimal invasive surgical approaches to the brain. However, successful therapy of the central nervous system requires new tools for delivery of therapeutics. Indeed, the application of therapeutic genes, proteins or compounds via pumps into the CSF was shown to be only of limited value since the genes, proteins or compounds are not sufficiently transported within the tissue or are simply not taken up by the neurons.

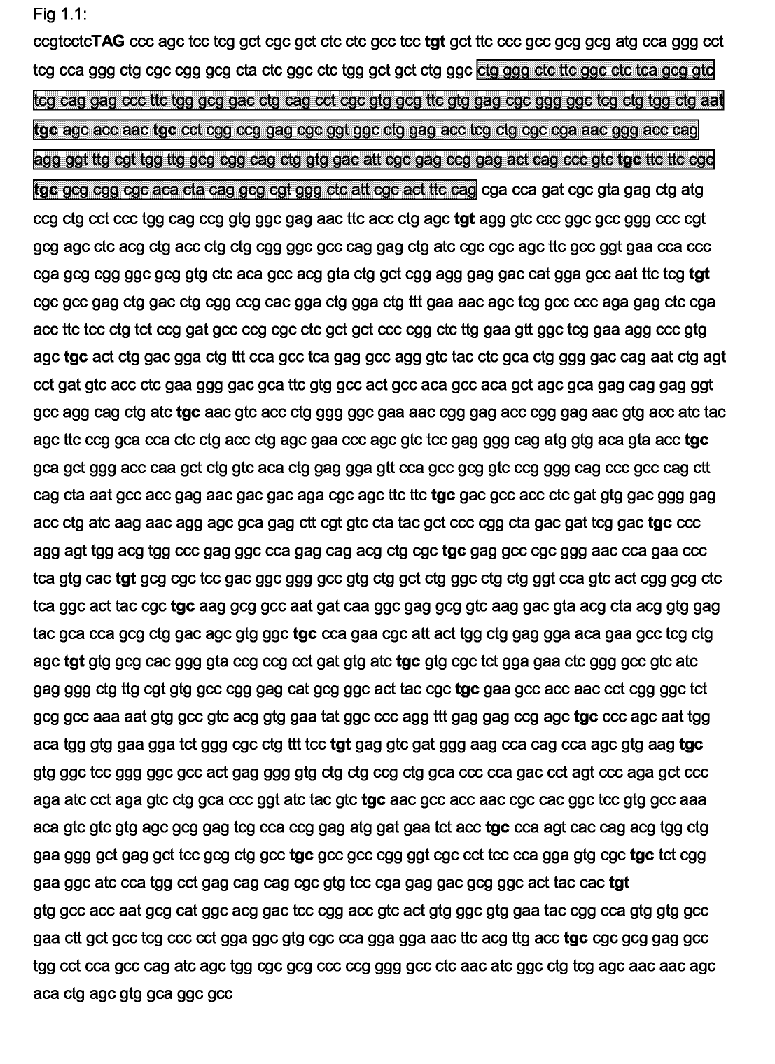

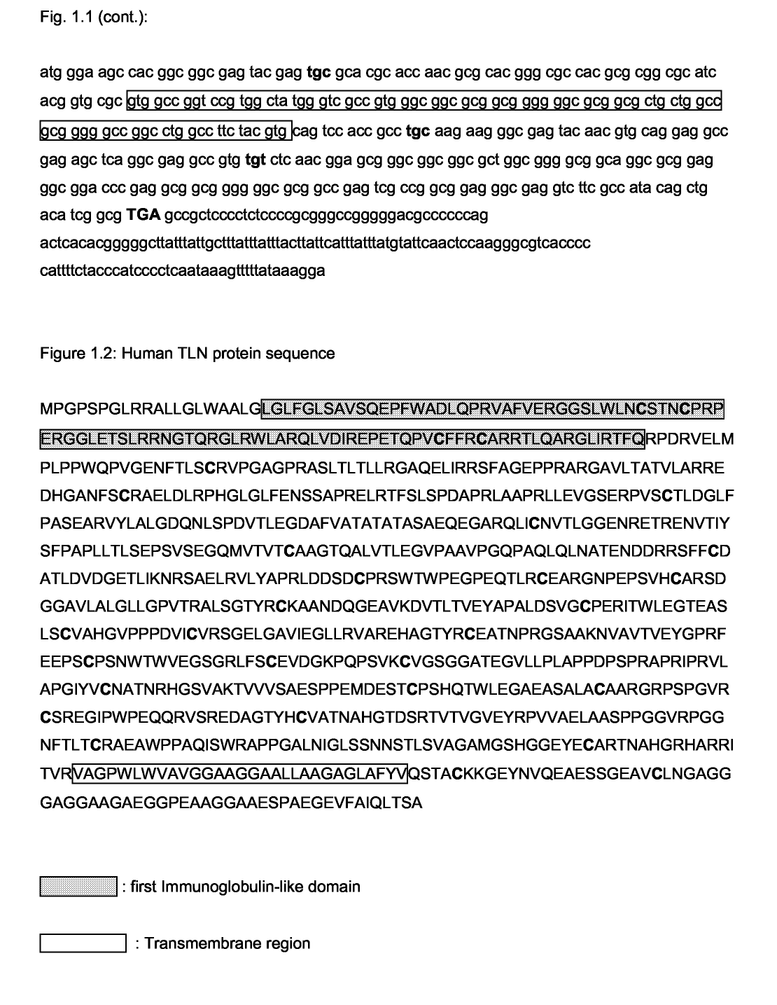

In the present invention we show for the first time that neurons are capable of performing phagocytosis. We further show that the process of phagocytosis is mediated by the transmembrane protein telencephalin (TLN, also designated as ICAM-5). For the sake of clarity the nucleic acid sequence and the amino acid sequence of human TLN is depicted in FIG. 1.

In a first embodiment the invention provides a method to modulate phagocytosis of neurons comprising modulating the expression of telencephalin expressed on said neurons wherein said modulation is a stimulation of phagocytosis via increased expression of telencephalin, or is a down-regulation of phagocytosis via a decreased expression or a decreased functionality of telencephalin. The word ‘modulation’ refers to the fact that phagocytosis can be enhanced (increased, up-regulated or stimulated are also equivalent terms here) or to the fact that phagocytosis can be down-regulated (decreased, suppressed or prevented are also equivalent terms here). With “down-regulating” it is understood that down-regulation can occur for at least 20%, 30%, 30%, 50%, 60%, 70%, 80%, 90% or even 100%. With “enhanced” it is understood that the enhancement can occur for at least 100%, 200%, 300%, 400% or even more. It is also envisaged here that the enhancement of phagocytosis refers to the fact that cells that are not capable of performing phagocytosis become capable of performing phagocytosis through a de novo expression of telencephalin in said cells. In yet another embodiment the invention provides a method to stimulate phagocytosis in cells wherein said stimulation occurs via gene transfer of telencephalin. Cells can be neuronal or non-neuronal cells. Thus one method to stimulate phagocytosis in cells is via gene transfer of telencephalin. Preferably, the stimulation of phagocytosis is in cells which are not capable of performing phagocytosis. Gene transfer of telencephalin involves the use of gene therapy to deliver a polynucleotide encoding telencephalin. Thus the present invention makes use of the nucleic acid of telencephalin for the transfection of cells in vitro and in vivo. This nucleic acid can be inserted into any of a number of well-known vectors for the transfection of target cells as described below. Target cells are preferably non-phagocytosing or very low-phagocytosing cells such as liver cells. The nucleic acid is transfected into cells, ex vivo or in vivo, through the interaction of the vector and the target cell. Said nucleic acid, under the control of a promoter, then expresses telencephalin, thereby mitigating the effects of absence or in some instances also the shortage of telencephalin. Delivery of the gene or genetic material into the cell is the first critical step in gene transfer or gene therapy. A large number of delivery methods are well known to those of skill in the art. Preferably, the nucleic acids are administered for in vivo or ex vivo gene transfer uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Flegner, WO 91/17424, WO 91/16024. Delivery can be to cells (ex vivo administration) or target tissues (in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); U.S. Pat. Nos. 4,186,183, 4,217,344, 4,235,871, 4,261,975, 4,485,054, 4,501,728, 4,774,085, 4,837,028, and 4,946,787). The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues. The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised on cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); PCT/US94/05700. In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., U.S. Pat. No. 4,797,368; WO 93/24641; Kotin, Human Gene Therapy 5:793-801 (1994); Muzyczka. Construction of recombinant AAV vectors are described in a number of publications, including U.S. Pat. No. 5,173,414; Hermonat & Muzyczka, Proc. Natl. Acad. Sci. U.S.A. 81:6466-6470 (1984); and Samulski et al., J. Virol. 63:03822-3828 (1989). In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94/22 12133-12138 (1997)); Pa317/pLASN was the first therapeutic vector used in a gene therapy trials. (Blaese et al., Science 270:475-480 (1995)). Transduction efficiencies of 50% greater have been observed for MFG-S packaged vectors (Ellem et al. Immunol Immunother. 44(1):10-20 (1997); Dranoff et al., Hum. Gene Ther. 1:111-2 (1997)). Recombinant adeno-associated virus vectors (rAAV) are a promising alternative gene delivery systems based on the defective and non-pathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998). Replication-deficient recombinant adenoviral vectors (Ad) are predominantly used in transient expression gene therapy, because they can be produced at high titer and they readily infect a number of different cell types. Most adenovirus vectors are engineered such that a transgene replaced the Ad E1a, E1b, and E3 genes; subsequently the replication deficient vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Sterman et al., Hum. Gene Ther. 9:7 1083-1089 (1998); Alvarez et al., Hum. Gene Ther. 5:597-613 (1997); Topf et al., Gene Ther. 5:507-513 (1998)). Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92/9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences thought to favour uptake by specific target cells. Gene therapy vectors can be delivered in vivo by administration to an individual patient, typically by systemic administration (e.g., intravenous, intra-peritoneal, intra-muscular, sub-dermal, or intra-cranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients). In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176: 1693-1702 (1992)). Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)). Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Administration is by any of the routes normally used for introducing a molecule into ultimate contact with blood or tissue cells. The nucleic acids are administered in any suitable manner, preferably with pharmaceutically acceptable carriers. Suitable methods of administering such nucleic acids are available and well known to those of skill in the art, and, although more than one route can be used to administer a particular composition, a particular route can often provide a more immediate and more effective reaction than another route.

In yet another embodiment the invention provides a method for down-regulating phagocytosis in neurons via the binding of a molecule to the nucleic acid encoding telencephalin or to the telencephalin protein wherein said molecule is chosen from the list consisting of an antibody (protein), an RNAi molecule (nucleic acid), or an anti-sense molecule (nucleic acid). The binding of such a molecule results in a decreased expression of telencephalin or in a decreased functionality of telencephalin. The wording “a decreased functionality of telencephalin” refers to the fact that a molecule interacting with telencephalin prevents its phagocytic function. By binding of such a molecule (e.g. an antibody) telencephalin is not capable anymore of recognizing a particle, complex or pathogen that can enter the neuron through phagocytosis.

Thus according to the invention molecules that comprise a region specifically binding to telencephalin or nucleic acids encoding telencephalin, which can be used for modulating the process of phagocytosis of neurons, are chosen from the list comprising an antibody or any fragment thereof binding to telencephalin, a (synthetic) peptide, a protein, a small molecule specifically binding to telencephalin or to nucleic acids encoding telencephalin or a regulatory region (e.g. a promoter region) of telencephalin, RNA aptamers against telencephalin, a ribozyme against nucleic acids encoding telencephalin, anti-sense nucleic acids hybridising with nucleic acids encoding telencephalin and small interference RNA's (siRNA) against telencephalin.

The wording ‘a molecule which comprises a region specifically binding to telencephalin or nucleic acids encoding telencephalin relates (1) on the one hand to molecules binding to nucleic acids encoding telencephalin or to regulatory genetic regions of telencephalin, said molecules inhibit the gene expression of telencephalin (thus the inhibition of telencephalin transcription and/or translation of a gene transcript (mRNA) of telencephalin and (2) on the other hand to molecules that inhibit the activity of the telencephalin protein. The inhibition of gene expression can be measured conveniently by methods known in the art such as for example RT-PCR analysis of the telencephalin transcript or for example western blot analysis of the telencephalin protein, said inhibition is preferably at least 20%, 30%, 40%, 50%, 60%, 70%, 80%, 90% or even higher. Measurement of molecules that bind to the telencephalin protein and inhibit the activity of telencephalin and hence phagocytosis can for example be carried out by various methods for determining phagocytosis as described in the examples of the present invention. Said inhibition of phagocytosis activity is preferably at least 20%, 30%, 40%, 50%, 60%, 70%, 80%, 90% or even higher.

The term ‘antibody’ or ‘antibodies’ relates to an antibody characterized as being specifically directed against telencephalin or any functional derivative thereof, with said antibodies being preferably monoclonal antibodies; or an antigen-binding fragment thereof, of the F(ab′)₂, F(ab) or single chain Fv type, or any type of recombinant antibody derived thereof. These antibodies of the invention, including specific polyclonal antisera prepared against telencephalin or any functional derivative thereof, have no cross-reactivity to others proteins. The monoclonal antibodies of the invention can for instance be produced by any hybridoma liable to be formed according to classical methods from splenic cells of an animal, particularly of a mouse or rat immunized against telencephalin or any functional derivative thereof, and of cells of a myeloma cell line, and to be selected by the ability of the hybridoma to produce the monoclonal antibodies recognizing telencephalin or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′₂ and ssFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type. In a specific embodiment the antibodies against telencephalin (also designated as antibodies with a specificity for telencephalin) can be derived from animals of the camelid family. In said family immunoglobulins devoid of light polypeptide chains are found. Heavy chain variable domain sequences derived from camelids are designated as VHH's. “Camelids” comprise old world camelids (Camelus bactrianus and Camelus dromaderius) and new world camelids (for example Lama paccos, Lama glama and Lama vicugna). EP0656946 describes the isolation and uses of camelid immunoglobulins and is incorporated herein by reference.

Small molecules, e.g. small organic molecules, and other drug candidates can be obtained, for example, from combinatorial and natural product libraries.

Also within the scope of the invention are oligoribonucleotide sequences, that include anti-sense RNA and DNA molecules and ribozymes that function to inhibit the translation of telencephalin mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of the telencephalin nucleotide sequence, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of telencephalin RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize anti-sense RNA constitutively or inducibly, depending on the promoter used, can be introduced stably into cell lines.

In a particular embodiment short interference RNA molecules (siRNA) can be used for the manufacture of a medicament for treatment of pathological angiogenesis. Said interference RNA molecules can be generated based on the genetic sequence of telencephalin (see FIG. 1). RNA interference (RNAi) is based on the degradation of particular target sequences by the design of short interference RNA oligo's (siRNA) which recognize the target sequence (the sequence of TLN is depicted in FIG. 1) and subsequently trigger their degradation by a poorly understood pathway. In general siRNA duplexes are shorter than 30 nucleotides, because longer stretches of dsRNA activate the PKR pathway in mammalian cells which results in a global a-specific shut-down of protein synthesis. The preparation and gene therapy vectors for the intracellular expression of siRNAs duplexes is disclosed in WO0244321 which is herein incorporated by reference.

In yet another embodiment an antibody with a specificity for telencephalin or a small molecule or anti-sense molecule binding to telencephalin or to the nucleic acid encoding telencephalin can be used for the preparation of a medicament to treat infectious diseases of neurons. With the wording ‘infectious diseases of neurons’ it is meant bacterial, viral and prion diseases of neurons. Infection of the central nervous system (CNS) is a severe and frequently fatal event during the course of many diseases caused by several pathogenic agents. One class of pathogenic agents consists of microbes with a predominant intracellular life cycle. Examples of these comprise the facultative intracellular bacteria Listeda monocytogenes, Mycobactedum tuberculosis, Brucella and Salmonella spp. and obligate intracellular microbes of the Rickettsiaceae family and Tropheryma whipplei. Other examples of bacteria comprise several bacterial species causing meningitis and Nocardia asteroides. Another class of pathogenic agents consists of viruses comprising Rabies virus (RABV), West Nile Virus, HIV virus, neurotropic herpes viruses and Sindbis virus. Still other infectious agents include also neuron infections by for example Mycoplasma.

The mechanisms used by these pathogenic agents to enter the CNS were not known but in the present invention we show that the transmembrane receptor telencephalin is an important entry molecule for infectious agents of neurons.

The term ‘medicament to treat’ relates to a composition comprising molecules as described herein above and a pharmaceutically acceptable carrier or excipient (both terms can be used interchangeably) to treat or to prevent infectious diseases of neurons. The administration of a molecule or a pharmaceutically acceptable salt thereof may be by way of oral, inhaled, topical, intra-cerebro-ventricular, intrathecal or parenteral administration. The active compound may be administered alone or preferably formulated as a pharmaceutical composition. An amount effective to treat infectious diseases of neurons depends on the usual factors such as the nature and severity of these infections being treated and the weight of the mammal. Doses will normally be administered continuously or once or more than once a day, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg. It is greatly preferred that the compound or a pharmaceutically acceptable salt thereof is administered in the form of a unit-dose composition, such as a unit dose oral, parenteral, topical or inhaled composition. Such compositions are prepared by admixture and are suitably adapted for oral, inhaled, topical, intra cerebro-ventricular, intrathecal or parenteral administration, and as such may be in the form of tablets, capsules, oral liquid preparations, powders, granules, ointments, lozenges, reconstitutable powders, injectable and infusable solutions or suspensions or suppositories or aerosols. Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art. Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate. These solid oral compositions may be prepared by conventional methods of blending, filling, tabletting or the like. Repeated blending operations may be used to distribute the active agent throughout those compositions employing large quantities of fillers. Such operations are, of course, conventional in the art. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents. Oral formulations also include conventional sustained release formulations, such as tablets or granules having an enteric coating. Preferably, compositions for inhalation are presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, for example between 1 and 5 microns, such as between 2 and 5 microns. A favored inhaled dose will be in the range of 0.05 to 2 mg, for example 0.05 to 0.5 mg, 0.1 to 1 mg or 0.5 to 2 mg. For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum. Parenteral suspensions are prepared in substantially the same manner except that the compound is suspended in the vehicle instead of being dissolved and sterilised by exposure to ethylene oxide before suspending in the sterile vehicle. Advantageously, a surfactant or wetting agent is included in the composition to facilitate uniform distribution of the active compound. Where appropriate, small amounts of bronchodilators for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. As is common practice, the compositions will usually be accompanied by written or printed directions for use in the medical treatment concerned. In a particular embodiment the ‘medicament’ may be administered by a method close to the place of onset. Preferably a continuous infusion is used and includes the continuous subcutaneous delivery via an osmotic minipump. In another embodiment said close to the onset administration is an intrathecal administration. In another embodiment said close to the onset administration is an intracerebroventricular administration. Intrathecal administration can for example be performed by means of surgically implanting a pump and running a catheter to the spine.

The present invention further provides a pharmaceutical composition for use in the treatment and/or prophylaxis of herein described infectious diseases of neurons which comprises a pharmaceutically acceptable salt thereof, or a pharmaceutically acceptable solvate thereof, and, if required, a pharmaceutically acceptable carrier thereof.

It will be understood that the appropriate dosage of the molecules should suitably be assessed by performing animal model tests, wherein the effective dose level and the toxic dose level as well as the lethal dose level are established in suitable and acceptable animal models. Further, if a substance has proven efficient in such animal tests, controlled clinical trials should be performed. Needless to state such clinical trials should be performed according to the standards of Good Clinical Practice.

In a particular embodiment the compounds of the invention can be used alone or in combination with other antibiotics such as erythromycin, tetracycline, macrolides, for example azithromycin and the cephalosporins. Depending on the mode of administration, the compounds will be formulated into suitable compositions to permit facile delivery to the affected areas.

Formulations may be prepared in a manner suitable for systemic administration or topical or local administration. Systemic formulations include those designed for injection (e. g., intramuscular, intravenous or subcutaneous injection) or may be prepared for transdermal, transmucosal, or oral administration. The formulation will generally include a diluent as well as, in some cases, adjuvants, buffers, preservatives and the like.

In yet another embodiment telencephalin is used as an entry site for compounds to enter cells expressing telencephalin comprising a) providing an antibody or a ligand for telencephalon and b) linking said antibody or ligand to a compound resulting in a complex and c) administrating said complex to cells expressing telencephalin resulting in phagocytosis of said complex. The wording “used as an entry site for compounds” refers to the fact that molecules or complexes can be directed specifically to cells expressing telencephalin. Preferably such cells are neurons, more preferably such cells are neurons residing in the telencephalon. Since the present invention shows that telencephalon is a receptor for phagocytosis the compounds or complexes directed to cells comprising telencephalon will be taken up by said cells through phagocytosis. In one aspect such compounds that can be specifically directed to cells, preferably neurons, are medicines (e.g. small natural or unnatural compounds, proteins, genes, siRNAs, ribozymes). In another aspect such compounds can be used for the development of a diagnostic assay. A compound can be directed to telencephalon through linking (or coupling) said compound with a ligand for telencephalon. Such a compound-ligand complex is herein designated as a complex. A ligand can be a natural ligand for telencephalin such as the leukocyte integrin CD11a (Tian L. et al (1997) J. Immunol 158(2):928-36). Alternatively a ligand is an unnatural ligand such as a molecule with a high specificity for telencephalon. In another embodiment a compound can be directed to telencephalon through coupling said compound to an antibody with specificity for telencephalon. In a preferred aspect an antibody with specificity for telencephalon is a camelid antibody. Such a camelid antibody can also be used to deliver siRNA duplex and also cDNA carrier vectors to cells comprising telencephalin. In the case of siRNA, chemically crosslinking can be achieved using thiol-modified siRNA duplexen and the antibody with specificity for telencephalin. For cDNA, vectors can be first packaged in liposomes. Second, the antibody with a specificity for TLN is modified to allow binding to PEG-maleimide-DSPE and this complex is transferred to liposomes. The methodology for making liposomes is described in Mastrobattista E. et al (2002) J. Biol. Chem. 277(30):27135-43, Ishida T. et al (1999) FEBS Letters 460(1):129-33. Uptake of antibody-coated loaded liposomes can be tested using the phagocytosis assay described herein. The effect of siRNA or cDNA administration to cells comprising telencephalon, such as neurons, can also be tested by SDS-PAGE and western blot analysis of the targeted protein(s). In a particular embodiment a compound (or glycoconjugate) can be made which can be used for the clearing of plaques comprising amyloid beta from diseased brain (e.g. Alzheimer's disease). An example of such a conjugate is described in example 13.

The present invention is further illustrated by the following examples which should not be construed as limiting in any way. The contents of all cited references (including literature references, issued patents, published patent applications, and co-pending patent applications) cited throughout this application are hereby expressly incorporated by reference.

### EXAMPLES

1. Introduction

Presenilin 1&2 (PS1&2) play a catalytic role in the γ-secretase complex needed for regulated intramembrane proteolysis (RIP) of a growing list of type I integral membrane proteins. This mediates a multitude of downstream signaling cascades, either by generating an intracellular domain that acts as a transcriptional (co-)factor or by regulating the availability of associated protein networks. We have documented before (Annaert et al., 2001) the interaction of PS1 with telencephalin (TLN), a neuron-specific intercellular cell adhesion molecule (ICAM-5) involved in dendritic outgrowth and long term potentiation. In PS1−/− hippocampal neurons we have observed that TLN accumulates in large somatic structures, coupling PS1 function to TLN localization and trafficking. Both the nature of these accumulations as well as the question whether they originate as a direct consequence of impaired y-secretase processing of TLN were studied here.

2. Telencephalin is Not Cleaved by γ-Secretase

In wild-type hippocampal neurons, TLN localizes to the plasmalemma whereas in 30% of PS1−/− neurons, TLN additionally accumulates in large somatic structures, the identity of which remained elusive (Annaert et al., 2001). APP, a substrate for γ-secretase, did not co-enrich in these structures suggesting that PS1 deficiency affects APP and TLN differentially. Therefore we tested whether TLN is a γ-secretase substrate. In 15 day old PS1−/− hippocampal cultures we could not detect any TLN C-terminal fragment (CTF) as apposed to APP-CTF that readily accumulate. Even upon Semliki Forest Virus (SFV)-induced overexpression, no TLN-CTF was detected. Since no putative mechanism of shedding of the TLN ectodomain is known, we generated a TLNΔE that could serve as a potential γ-secretase substrate. No intracellular domain fragments were however detected indicating the absence of γ-secretase cleavage. Since this could reflect a fast turnover of the intracellular domain after cleavage, as seen for Notch and APP, we used a highly sensitive reporter assay (Struhl and Adachi, 2000) by coupling the Gal4VP16 transactivator to the cytoplasmic domain of TLN. Release of a potential intracellular domain of TLN can be measured via the activity of luciferase. While for APP and Notch nuclear translocation of the inducer fragment was prominent, and strongly inhibited by different γ-secretase inhibitors (125 mM DAPT, L685,458 or compound C), no signal could be detected with TLNΔE-Gal4VP16. To investigate whether the TLN transmembrane domain could be a γ-secretase substrate in the context of the APP protein, we swapped the transmembrane regions of TLN and APP to create an APP/TLN_{TMR}-chimaera. Even in this case no activity could be detected. Therefore we conclude that TLN is most likely not a γ-secretase substratum.

Finally, we investigated to what extent the TLN accumulations depended on γ-secretase activity. Treatment of wild-type neurons with γ-secretase inhibitors for one week (daily administered from day 7 till 14) did not induce aberrant TLN accumulations although γ-secretase cleavage of APP-CTF was markedly inhibited.

Of notice, in 25 d-old PS1−/− neurons the frequency of TLN accumulations did not further increase. Instead, in individual neurons they tended to be more numerous (grape-like) or larger (suggesting they may undergo fusion). These accumulations were not encountered in PS1± neurons nor in neurons expressing human PS1 in a PS1−/− background. A true rescue was however achieved by re-introducing human PS1 using adenoviral infection. Increasing the MOI (multiplicity-of-infection) from 250 to 6,000 virus particles/neuron suppressed the frequency of TLN accumulations to wild-type level. Importantly, similar rescue effects were obtained with FAD-linked G384A- or D257A-dominant negative PS1 mutants arguing again for a γ-secretase independent effect of PS1. Since TLN accumulations only appeared in ±30% of PS1−/− neurons, this full rescue could only be achieved with a very high transfection efficiency. Control adenoviral expression of eGFP indeed resulted in an 85-95% efficiency. Moreover, protein expression lasted up to 11 days post-infection as shown for PS1. In summary, exogenous expression of wild-type or mutant PS1 is sufficient to restore the aberrant TLN phenotype.

3. The Turnover of Full-Length TLN is Affected in PS1−/− Neurons

As PS1 is abundantly localized in pre-Golgi compartments TLN accumulations may reflect a transport block in the early secretory pathway due to the absence of PS1. We tested this by analyzing the glycosylation kinetics of TLN. We overexpressed TLN using the SFV-system in wild-type and PS1−/− cortical neurons and performed pulse-chase experiments in combination with endoH treatment to quantify the ratio of mature to immature glycosylated TLN. Phosphorimaging analysis revealed no statistical differences in this ratio indicating that transport kinetics of newly synthetized TLN are similar in wild-type and PS1−/− neurons, as was seen for APP. However, using the same pulse-chase paradigm, we found that the half-life of newly synthesized TLN, but not full-length APP, was delayed in PS1−/− neurons. If turnover is delayed, this should be reflected in a relative increase of endogenous TLN in PS1−/− neurons. Indeed, western blotting and densitometric analysis showed an almost 4-fold increase of TLN compared to wild-type neurons. Interestingly, TLN appeared to be endoglycosidase H resistant suggesting that TLN accumulation occurs in post-Golgi compartments (see below). Further controls indicated a weak accumulation of full-length APP and an impressive 44-fold increase in APP-CTF.

4. TLN Accumulations do Not co-Distribute with ‘Classical’ Early and Late Compartments

To understand the possible mechanism(s) behind the increased protein levels and delayed turnover, we set out to identify the compartment where TLN accumulates in PS1−/− neurons. First, TLN accumulations could not be identified as nuclear inclusions and essentially no overlap was detected with marker proteins of the endoplasmic reticulum (ER) such as BIP and calnexin. Other compartments of the early secretory pathway, including the intermediate compartment (ERGIC-53) and Golgi apparatus (62 -COP and GM130) were equally devoid of TLN immunostaining. Although TLN accumulations are found close beneath the cell surface they were not accessible to exogenous biotin. We detected no association with early (EEA1) or recycling endosomes (transferrin receptor) nor with late endosomes (LBPA) and lysosomes (Lamp-2 and cathepsin D (catD)).

These findings encouraged us to implement electron microscopy (EM). Due to the specifications of the culture, we applied a new flat-embedding technique that preserves the in situ orientation of polarized neurons (Oorschot et al., 2002) and combined this with correlative light-immunoEM (Koster and Klumperman, 2003). This allowed us to localize TLN accumulations prior to EM processing. ImmunoEM revealed abundant gold label at the plasmalemma and in large membrane-bound vacuoles. In these structures label was found both on the limiting membrane and internal membranes. Interestingly, LAMP-1 labeled lysosomes but not TLN positive structures confirming that they are distinct from pre- or lysosomal compartments. Except for their large size, their heterogeneous content including tubular and vesicular structures is suggestive for an autophagic origin.

5. Characterization of TLN-Positive Autophagic Vacuole-Like Structures

Autophagy is the intracellular bulk degradation system and plays an important role in the cellular protein economy. To further identify TLN-positive structures, we first studied the uptake of monodansylcadaverine (MDC) a compound used to identify autophagic vacuoles. MDC co-accumulated with TLN in PS1−/− neurons. Noteworthy, MDC uptake occurred in the absence of starvation indicating that TLN accumulations are pre-existing structures. Autophagic vacuoles originate from an elongation process of isolation membranes that close and finally fuse with late endosomes/lysosomes. Several ubiquitin-like conjugation systems are essential for autophagic vacuole formation. Among them, the Apg12p-Apg5p conjugate is important in the initial steps where it localizes (albeit a small fraction) to the outerside of the isolation membrane throughout the elongation process and dissociates again upon completion of the autophagic vacuole. A second protein is LC3, that remains partially on the autophagic vacuole even after fusion with lysosomes. In PS1−/− neurons, both anti-Apg12p and -LC3 antibodies immunostained TLN accumulations. However, Apg12p remains stably associated with these structures which is surprising since this protein is reported to dissociate before or upon closure of the autophagic vacuole. This suggests that the normal formation and maturation of this type of autophagic vacuole is impaired in PS1−/− neurons. In wild-type neurons no colocalization of Apg12p and LC3 with TLN was observed demonstrating the unique nature of the accumulations. Interestingly, in some PS1−/− neurons small TLN- and Apg12p-positive structures were detected in the proximal regions of dendrites, possibly representing earlier stages of accumulation.

6. TLN Localizes to Autophagic Vacuoles in catD−/− Hippocampal Neurons

These findings prompted us to investigate in more detail the relationship of TLN with autophagic vacuoles. In many cases autophagic vacuole accumulation is associated with defective lysosomal biogenesis. In our case, however, no difference in catD maturation was found between wild-type and PS1−/− neurons and lysosomal delivery of catD is therefore not impaired. On the other hand, catD deficiency results in the accumulation of autophagic vacuoles/autophagosomes. This is also true in primary hippocampal neurons derived from catD−/− embryos as can be observed with Lysotracker. Although TLN accumulations are clearly not acidified in PS1−/− neurons, acidic organelles were often found in close apposition and likely represent lysosomes. In catD−/− neurons, some TLN immunoreactivity was detected in the large Lysotracker-positive organelles. Ultrastructurally, these TLN-positive organelles resemble dense autophagic vacuole-like structures. Importantly, these organelles are smaller in diameter compared to TLN-accumulations and were not seen in wild-type or PS1−/− neurons. Taken together, in catD−/− neurons TLN localizes to autophagic vacuoles suggesting that these organelles are part of the normal physiological route for TLN degradation.

7. TLN Mediates Phagocytic Uptake of Microbeads in Primary Hippocampal Neurons

We have shown that TLN accumulations do not share endosomal/lysosomal components. Also, TLN normally localizes to the somatodendritic plasmamembrane and accumulates as a mature protein suggesting that accumulations originate from the plasmamembrane through an internalization event distinct from endocytosis. To test whether phagocytosis is involved, we triggered this process by challenging hippocampal neurons with 2 μm microbeads. Already after 4 hrs, surprisingly many beads were found associated with neurites, and recruited TLN immunoreactivity. Longer incubations (24 or 48 hrs) resulted in a complete redistribution of TLN to microbeads. Since actin polymerization is considered the driving force of phagosome cup formation, we co-stained with phalloidin-Alexa568. Actin polymerization clearly co-localizes in a ring- or cup-shaped pattern with TLN on individual microbeads. Although the exact mechanism in phagosome formation is not clear, regulation by local phosphoinositide production is crucial in actin polymerization. Indeed, and next to actin, also endogenous phosphatidylinositol biphosphate (PIP2) is recruited to the TLN-positive phagosomal cup. Interestingly, PIP2 also co-localized to discrete TLN spots probably representing normal plasmamembrane localization of TLN. Notably, levels of PIP2 between different TLN-positive microbeads were sometimes very variable. This may reflect different stages of phagosome formation since PIP2 is rapidly lost upon phagosome sealing and completion due to the recruitment of phospholipase C. In agreement with this, no or almost no PIP2 was detected on TLN accumulations in PS1−/− neurons. Microbead uptake was equally observed both in wild-type and PS1−/− neurons indicating that this phagocytic process does not require PS1. Our data show that phagocytosis occurs also in neurons and that TLN is involved in this process.

8. Induction of Phagocytosis in Non-Phagocyting Cells

HeLa cells are not capable of performing phagocytosis. Furthermore HeLa cells do not express TLN. We tested if we could induce phagocytosis in HeLa cells via gene transfer of TLN to these cells. HeLa cells were plated out on coverslips and transfected with an expression vector containing full length TLN (pSG5**FL_TLN). Twenty-four hours post-transfection, microbeads were added. These beads varied in size from 0.5 to 1.0 and 2.0 μm and were incubated with the transfected cells overnight. A shorter incubation time of several hours proved as effective in the uptake assay. Cells were subsequently fixed in paraformaldehyde and following permeabilization, stained for TLN (using antibodies PAb B36.1) and actin (using labeled phalloidin). Actin localization was determined as its polymerization is known to mediate fagocytic uptake of particles, including microbeads. Phase contrast (DIC, differential interference contrast) was used to localize the microbeads. Alternatively, microbeads can also be used that are fluorescently labelled to allow a more easy detection. Localization is assayed by confocal laser scanning microscopy. Surprisingly the analysis showed that microbeads were only associated with HeLa cells that expressed TLN. TLN was shown to localize to the beads often in a ring-shape around the beads. Actin was similarly shown to localize around the beads, co-localizing with TLN. Few or no beads were associated with HeLa cells that were not transfected (no TLN expression). This assay is now used to quantify the capacity of TLN to phagocytose microbeads by measuring the ratio of TLN-transfected over microbead-containing HeLa cells. With this assay the effect of molecules binding to TLN or a nucleic acid encoding TLN (for instance antibodies with a specificity for TLN) can be easily scored (blocking the uptake of microbeads). Experiments are also designed to transfect HeLa cells with truncation or deletion mutants of TLN followed by microbead uptake in order to further fine map the region within the first lg-like domain of TLN that is required for phagocytosis.

9. Recombinant Production of TLN

TLN (ICAM-5) interacts with LFA-1/CD11a-CD18 integrin via a short region in the first immunoglobulin-like (Ig-like) domain of TLN. It is shown in the art that antibodies directed against this domain block this interaction. The same region also binds T lymphocytes, probably mediated through the same integrin on these cells. We generate antibodies that recognize the first Ig-like domain since this domain is involved in the phagocytic uptake mechanism. This domain is encoded by amino acids 21 to 118 of the human TLN sequence (see FIG. 1: grey boxed area in protein sequence, and the corresponding cDNA sequence of human TLN). Since this domain is posttranslationally modified by glycosylation and has disulfide bonds for the assembly of a functional domain, the antigen is prepared by expressing the cDNA comprising this region, fused to a tag (HIS, GST, Fc, etc) in a mammalian cell line. For this purpose, the corresponding cDNA is cloned in an eukaryotic expression vector (pcDNA3.1 and pSG5) in frame with the sequence encoding HIS, GST or Fc. These vectors are used to transfect COS7 cells. Recombinant domains are collected from the conditioned media and affinity purified on nickel-Sepharose (His-tag), glutathione-Sepharose (GST) or proteinA-Sepharose (Fc). Purified antigen is coupled to an appropriate carrier (e.g. KLH) and used for immunization.

10. Generation of Camel Antibodies Directed Against TLN

The purified TLN (see example 9) is used to immunize a camel. After immunisation, the VHH repertoire of an immunized camel is cloned in phage display vectors to select the antigen-specific VHHs from these ‘immune VHH libraries’ (Nguyen et al. (2003) Immunology 109, 93). Once specific VHHs are selected they will be fluorescently tagged (according to established procedures in the art and commercially available kits) and tested by immunocytochemical techniques for its binding to endogenous TLN in hippocampal neurons or in mouse embryonic fibroblasts or HeLa cells stably or transiently expressing exogenous TLN.

11. Prevention of Bacterial Pathogen Infection of Neurons

Primary hippocampal neurons are routinely and highly reproducibly prepared according to established procedures. In general, this neuronal model systems consists of a highly homogeneous population of pyramidal neurons (95% purity) that all express TLN. Differentiated neurons (day 7-12 post-plating) are challenged with either fluorescently labeled microbeads or commercially available bioparticles (E. coli or S. aureus). Uptake of particles in neurons is measured and quantified using the below described phagocytosis assay (see Materials and Methods, section 7). To study the function of TLN in mediating phagocytic uptake, TLN protein expression is downregulated using selected anti-TLN camellid antibodies that block phagocytic uptake or by use of a siRNA against TLN. For this, 3 day old neurons can be transfected with siRNA duplexes using commercial transfection agents (Lipofectamine2000 or Fugene6).

12. Use of Inhibitors of Telencephalin to Inhibit Neuronal Infection of Neurotrophic Viruses

The uptake of the neurotrophic herpes simplex is studied in TLN-expressing neurons in the brain. The first cellular model system are primary hippocampal neurons that express TLN and primary neurons that do not expresss TLN. They comprise either mixed cultures of cortical neurons, inhibitory GABAergic neurons that comprise a 10% population in a routine primary hippocampal neuron culture and finally primary hippocampal neurons derived from the hippocampi of TLN knock-out mice. A second cellular model is the epithelial MDCK cell line, a representative cell line for polarized transport. This cell line is used for exogenous expression full length TLN, but is also used to produce TLN variants that are needed to demonstrate specificity and/or regulation of the pathogen uptake mechanism. The TLN variants include TLN that lacks the intracellular domain, TLN that bears a modified intracellular domain that abrogates interaction with downstream events such as the interaction with actin and actin-binding proteins, mutant TLN that has lost its binding capacity to LFA-1 or CD11a/CD18 integrins. A mutant TLN can be either a TLN with a deletion in the ectodomain or single amino acid substitutions.

Primary neurons or TLN-expressing MDCK cells are challenged with different concentrations (MOI) of viruses over different periods of time. Herpes Simplex virus is made visible by the integration of a fluorescent protein in its genome. Uptake of pathogen is measured and compared in both TLN expressing and non-expressing primary neurons and in MDCK cells. The uptake of pathogen is quantified by fluorescence. A second quantification method is SDS-PAGE of a cellular lysate followed by western blotting to detect the fluorescent antigen or a pathogen-specific glycoprotein of the viral envelope of HSV. Inhibition and/or specificity of uptake is tested by comparing the uptake in TLN-expressing and non-expressing primary neurons or using MDCK cell lines expressing modified -this can be truncated, deleted, or point mutated-TLN. Detection of exogenously expressed TLN is facilitated by the introduction of a fluorescent protein in the ectodomain or intracellular domain of TLN. Introduction of TLN in MDCK cells is performed using commercial transfection reagentia and/or electroporation. Stable integration of TLN in MDCK cells is acquired through retroviral transduction using the TLN cDNA (including modified cDNA's) cloned in pMSV vector. Production of retrovirus is done in Hek293 cells. Uptake of pathogen is inhibited by competing with the TLN ectodomain interaction using a recombinant TLN-binding domain in LFA-1. This binding domain is located within the ectodomain of LFA-1 and comprises 200 amino acids, named the I-domain. This I-domain is produced by overexpression of the corresponding cDNA in Sf2 insect cells using the commercial baculovirus system. The signal peptide sequence of LFA-1 is included in the fragment as well as a HIS-tag. The signal peptide sequence is needed to generate a secreted recombinant fragment that is collected from culture supernatants. The HIS-tag is required for a one-step affinity purification of the fragment from culture supernatant using commercial Nickel-Sepharose columns.

13. Clearing of Plaques in Mouse Models of Alzheimer's Disease

Telencephalon can be used as an entry site for a compound (or a conjugate) that is able to clear plaques comprising amyloid beta in the diseased brain. For the development of this conjugate one needs at least a bi-specific conjugate that comprises two binding elements: (1) an element binding specifically to amyloid beta (e.g. an antibody or a specific binding partner of amyloid beta and (2) an element specifically binding to TLN to be used as an entry site (e.g. an antibody against TLN or a ligand of TLN). A tri-specific conjugate comprising a third binding element is preferred. Said tri-specific conjugate comprising a third binding element is capable of binding to proteins present on cerebromicrovascular endothelial cells and wherein upon said binding transcytosis (thus crossing of the blood-brain barrier) occurs (e.g. antibodies to the human transferrin receptor are known to cross the blood-brain barrier, antibodies (e.g. derived from camels) are preferentially used as a third binding element for inducing the passage of the blood-brain barrier, examples of said antibodies are fully described in WO02/057445 (National Research Council of Canada). The scheme of the strategy is outlined below.

(1) An antibody is generated against amyloid β peptides. The selected epitope to which antibodies are generated is preferentially an epitope on amyloid β aggregates that is identical amongst amyloid β peptides. The common epitope is the so-called cross-β structure that is generated only when amyloid β-peptides convert to their oligomeric or fibrous conformation. Since oligomeric amyloid β is rather to become the culprit in the pathology, antibodies that recognize this structure are preferred. Oligomeric amyloid β is therefore used as the antigen for immunization of camels/lama's with the purpose to generate camelid antibodies. Oligomeric amyloid β, comprising amino acids 1-40 or 1-42, is naturally occurring and can for example be isolated from culture supernatants (Walsh, D., Klyubin, I., Fadeeva, J., Cullen, W., Anwyl, R., Wolfe, M., Rowan, M., Selkoe, D. Nature, 2002, vol. 416, p. 535-539). Alternatively, oligomeric or fibrous amyloid β can be generated from recombinant peptides only by incubating the solubilized peptide. Recombinant peptides are commercially purchased. After immunisation, the VHH repertoire of an immunized camel/lama is cloned in phage display vectors to select the antigen-specific VHHs, thus VHHs that recognize oligomeric amyloid β, from these ‘immune VHH libraries’.

As an alternative to use an antibody against amyloid beta as a first binding element, a domain of tPA can also be used as a first binding element. Indeed, tissue-type plasminogen activator (tPA) is a protease that has been demonstrated to bind specifically to amyloid β polypeptides and is a cross-β structure receptor. tPA or more selectively, the binding domain within tPA that binds cross-p structures (Kranenburg, O., Bouma, B., Kroon-Batenburg, L., Reijerkerk, A., Wu, Y., Voest, E., Gebbink, M. Current Biology, 2002, vol 12, p. 1833-1839: Tissue-type plasminogen activator is a multiligand cross-beta structure receptor) can be used as an alternative to an antibody against amyloid beta.
(2) In a next step the first binding element (e.g. the antibody against amyloid beta or the tPA binding domain) is fused to the TLN-binding domain in LFA-1 or CD11a/CD18. Fusion is accomplished either by chemical coupling of purified components or by cloning the cDNA encoding the TLN-binding domain in frame with the cDNA encoding the selected nanobody or tPA binding domain in an eukaryotic expression vector. In a preferred strategy a bi-specific antibody is made with one specificity for amyloid beta and another specificity for TLN. The generation of an antibody against TLN is described in example 10.

A bi-specific conjugate with elements (1) and (2) ((1)=a binding element against amyloid beta oligomers; (2)=a binding element against TLN) can for example be tested in MDCK cells that stably express TLN. In a next step this bi-specific conjugate is tested in plaque-forming mice by stereotactic injection in the hippocampal area. Single and repetitive injections are compared for their effectiveness to prevent plaque formation in the brain. Plaque-forming mice models include double and triple transgenic models harboring either FAD-linked PS1 and APP or FAD-linked PS1(M146V), APPswe and Tau (P301L) transgenes respectively. Plaque forming Alzheimer disease models of mice are well known in the art (e.g. Oddo, S., Caccamo, A., Shepherd, J., Murphy, P., Golde, T. Kayed, R., Metherata, R., Mattson, M., Akbari, Y., LaFerla, F., Neuron, 2003, Vol. 39, pp 409-421).

(3) The bi-specific conjugate as described above is preferentially coupled with a third binding element to generate a tri-specific conjugate. Said third binding element has a specificity for a protein present on cerebromicrovascular endothelial cells (for examples see the description above). The transcytosis of the resulting tri-specific binding conjugate is first evaluated in an in vitro model system for blood-brain barrier consisting of a co-culture of endothelial cells and glial cells. In a next step the tri-specific conjugate is used to prevent and/or to clean amyloid plaques in Alzheimer's disease mice models. Administration is for example via intravenous injection.

### Materials and Methods

1. Cell Culture Media and Antibodies

Cell culture media were from GibcoBRL. TOPRO-3 and Phalloidin-alexa568 (Molecular Probes) were used to label nuclei and actin. MDC was from Sigma. γ-Secretase inhibitors were from Calbiochem (X or L685,458), Elan (DAPT) and AstraZeneca (Compound C). Polyclonal anti-PS1-NTF (B19.2), -CTF (B32.1) and -TLN (B36.1) have been described (Annaert et al., 2001). B63.1 and B59.1 were generated using a synthetic peptide mimicking the final 16 and 18 amino acids of APP and nicastrin, respectively, coupled to KLH (Pierce). Mab 9C3 against nicastrin was produced by immunizing the same peptide in balb/c mice followed by generation of a hybridoma cell line according to established procedures. We acknowledge the antibody gifts of: anti-calnexin (A. Helenius, CH), anti-ergic-53 (J. Saraste, Norway) -LC3 (T. Yoshimori, Japan), -Apg12 (N. Mizoshima, Japan), PIP2 (G. Hammond, UK), -LBPA (J. Gruenberg, CH), -APP C-terminus (c 1/6.1, P. Mathews, N.Y.). Mabs to Lamp-2 (Abl-93) were obtained from DSHB (Iowa); anti-synaptophysin (cl.7.2) and anti-PS1-CTF (mab 5.2) were from R. Jahn (Germany) and B. Cordell (Scios, Calif.). Mabs to GM130 and EEA1 were from BD biosciences, the transferrin receptor from Zymed, β-COP from Sigma (Belgium), and BIP from Stressgen (Calif.).

2. Constructs and γ-Secretase Luciferase Assay

pSFV constructs encoding human APP, murine TLN and TLNΔE have been described (Annaert et al., 2001). APP and NotchΔE were cloned into the EcoRV site in front of the Gal4VP16 sequence in pIPAdApt vector. A construct encoding 10 aa of the ectodomain, the transmembrane and cytosolic domains of TLN was obtained by PCR and ligated in pGEMT containing the TLN signal peptide (SP). SP-TLNΔE was next cloned into pIPAdApt and GAL 4VP16 was inserted resulting in a pIPAdApt-SP-TLNΔE-Gal4-VP16 construct. For the APP/TLN_{TMR} chimaera, the coding region for APP-TMR was replaced by a PCR fragment encoding the TLN-TMR. Luciferase assay. Hela cells were transfected with 200 ng pFRluc plasmid (UAS-responsive luciferase construct, Stratagene) and 200 ng inducer plasmid using Fugene (Roche) After 24 cells were incubated with or without inhibitors (125 nM) and after 16 hrs lysed and assayed (Victor2, PerkinElmer).

3. Primary Neuron Cultures and Metabolic Labeling

HumanPS1 (in a PS1−/− background (Qian et al., 1998)), wild-type, PS1±, PS1−/− or catD−/− primary hippocampal neurons were derived from E17 embryos out of heterozygous crosses and co-cultured with a glial feeder layer (Annaert et al., 1999; Goslin and Banker, 1991). For metabolic pulse-chase labeling, mixed cortical neuron cultures from wild-type and PS1−/− were prepared (Annaert et al., 1999; De Strooper et al., 1998). At the end neurons were extracted and immunoprecipitated fractions were treated with 1OmU endopeptidase H (endoH, Boehringer) prior to SDS-PAGE (NuPAGE, Invitrogen) and phosphorimaging (Typhoon, Perkin Elmer). For western blotting, 10-12 day old hippocampal cultures were harvested in PBS, pelleted, resuspended in sample buffer and separated on SDS-PAGE. Blots were detected using chemiluminiscence (WesternLite, Perkin Elmer) and scanned using an internal standard (ImageScanner and TotalLab, AmershamPharmacia).

4. Adenoviral Infection

CDNAs of human wild-type, familial Alzheimer's disease-linked G384A or a dominant-negative D257A mutant PS1 were constructed into the pIPspADApt6 adapter plasmid which contains part of the adenoviral genome. CDNA encoding eGFP was used as a control. Adenoviruses were generated by co-transfection with helper cosmid DNA in the PER.C6/E2A adenoviral packaging cells and titers were determined (Michiels et al., 2002). For rescue experiments, PS1−/− hippocampal neurons grown for 4 to 6 days on coverslips (at a density of 800 to 1,200 neurons/coverslip) were infected overnight with different viruses at different MOls (250 to 6000) and returned to conditioned medium until fixation on day 15.

5. Confocal Laser Scanning Microscopy

Primary hippocampal neurons (14-25 days) were fixed in 4% paraformaldehyde/4% sucrose in 0.1M phosphate buffer (30 min, RT), permeabilized by methanol/aceton (−20° C.), 0.5% Triton X100/PBS or 0.5% saponin (5 min), and processed for indirect immunofluorescence (Annaert et al., 2001). Alexa 488- and 568-conjugated secondary antibody (Molecular Probes) stainings were detected through a Diaphot300 (Nikon) connected to a MRC1024 confocal microscope (BioRad). Data were collected, using Lasersharp3.0 and processed in Photoshop 7.0 (Adobe, Calif.). For double immunocytochemistry using pabs of the same host species blocked and permeabilized neurons were incubated overnight with biotinylated anti-TLN (B36.1). After blocking free sites with unconjugated Fab fragments (goat anti-rabbit, Jackson ImmunoResearch) coverslips were incubated (1 hr) with Alexa488-conjugated streptavidin (Molecular Probes) followed by second primary antibody and Alexa 568-conjugated goat anti-rabbit. In some cases neurons were incubated with biotin (Pierce), Lysotracker (Molecular Probes) or 50 mM MDC for 30 min at 4° C. or 37° C. prior to fixation and counterstaining with anti-TLN. For MDC, analysis was done on a Leica DMRB microscope equipped with UV-detection filter set (excitation wavelength 380 nm, emission filter 525 nm), a CCD camera (Photometrics ltd, Tucson, Ariz.) and QuipsFISH software (Vysis). To study phagocytic uptake, 14d neurons were incubated (4-48 hrs) with 2 μm microbeads (Polysciences, 50/cell) in conditioned medium, briefly washed, fixed and processed for immunocytochemistry.

6. ImmunoEM

Fixed neurons were quenched (50 mM NH4Cl, 5 min) and briefly permeabilized in 0.2% Triton X100 followed by incubation with B36.1. After washing, cells were incubated with Alexa488-conjugated goat anti-rabbit IgG to identify TLN-accumulations. Positively identified neurons were further processed for immunoEM using a flat embedding technique (Koster and Klumperman, 2003; Oorschot et al., 2002). The resulting ultrathin cryosections were labeled with anti-Alexa488 IgG (Molecular Probes) according to the protein A gold technique. Alternatively, cells were labeled with B36.1 and anti-Lamp-1, scraped and incubated with proteinA-gold (Slot et al., 1991).

7. Assay for Phagocysis

The process of phagocytosis is quantitated in cells by analyzing the internalization of a foreign particle, e.g. a fluorescently labeled immune complex, antibody, microbead or bacterial particles. The assay distinguishes the phagocytosed from non-phagocytosed particles using a fluorescence quenching assay with trypan blue. This technique takes advantage of the detectability of the fluorescently labeled phagocytosed particle while the fluorescence of the non-phagocytosed particle is quenched by trypan blue. Briefly, cells (MEFs, HeLa or primary neurons) are plated out in 96-well plates and are allowed to adhere (or differentiate in the case of neurons). Next, cells are incubated for different time intervals with the appropriate amounts of fluorescently labeled microbeads E. coli (K-12 strain) bioparticles (commercially available). Then, the medium is removed and exchanged with a trypan blue suspension for quenching. Microplates are analysed in a plate reader using the appropriate excitation and emission wavelengths. The net phagocytosis event is finally calculated. Using this approach in combination with the phagocytosis assay, we can select camellid antibodies against TLN for their ability to (i) block uptake of microbeads or pathogens or (ii) mediate uptake via phagocytosis.

8. Miscellaneous

Microsomal fractions of corteces of E17 wild-type and catD± and −/− embryos were either analyzed by westernblotting or assayed for γ-secretase activity using a cell-free assay. Briefly, 2% CHAPS extracts were incubated overnight with recombinant APP-C99 and newly produced Aβ was detected by western blotting (Nyabi et al., 2003).

### REFERENCES

Annaert, W. G., L. Levesque, K. Craessaerts, I. Dierinck, G. Snellings, D. Westaway, P. StGeorge-Hyslop, B. Cordell, P. Fraser, and B. De Strooper. 1999. Presenilin 1 controls gamma-secretase processing of the amyloid precursor protein in pre-golgi compartments of hippocampal neurons. J Cell Biol. 147:277-294.
Annaert, W. G., C. Esselens, V. Baert, C. Boeve, G. Snellings, P. Cupers, K. Craessaerts, and B. De Strooper. 2001. Interaction with telencephalin and the amyloid precursor protein predicts a ring structure for presenilins. Neuron. 32:579-89.
De Strooper, B., P. Saftig, K. Craessaerts, H. Vanderstichele, G. Guhde, W. Annaert, K. Von Figura, and F. Van Leuven. 1998. Deficiency of presenilin-1 inhibits the normal cleavage of amyloid precursor protein. Nature. 391:387-90.
Goslin, K., and G.A. Banker. 1991. Rat hippocampal neurons in low density culture. In Culturing Nerve Cells. K. Goslin, editor. MIT Press, Cambridge, Mass. 251-81.
Koster, A. J., and J. Klumperman. 2003. Electron microscopy in cell biology: integrating structure and function. Nature Cell Biology/Nature Reviews Molecular Cell Biology, suppl Imaging in Cell Biology: SS6-SS 10.
Michiels, F., H. van Es, L. van Rompaey, P. Merchiers, B. Francken, K. Pittois, J. van der Schueren, R. Brys, J. Vandersmissen, F. Beirinckx, S. Herman, K. Dokic, H. Klaassen, E. Narinx, A. Hagers, W. Laenen, I. Piest, H. Paviiska, Y. Rombout, E. Langemeijer, L. Ma, C. Schipper, M. D. Raeymaeker, S. Schweicher, M. Jans, K. van Beeck, I. R. Tsang, O. van de Stolpe, and P. Tomme. 2002. Arrayed adenoviral expression libraries for functional screening. Nat Biotechnol. 20:1154-7.
Nyabi, O., M. Bentahir, K. Horre, A. Herreman, N. Gottardi-Littell, C. Van Broeckhoven, P. Merchiers, K. Spittaels, W. Annaert, and B. De Strooper. 2003. Presenilins mutated at Asp-257 or Asp-385 restore Pen-2 expression and Nicastrin glycosylation but remain catalytically inactive in the absence of wild type Presenilin. J Biol Chem. 278:43430-6.
Oorschot, V., H. De Wit, W. G. Annaert, and J. Klumperman. 2002. A Novel Flat-embedding Method to Prepare Ultrathin Cryosections from Cultured Cells in Their In Situ Orientation. J Histochem Cytochem. 50:1067-80.
Struhl, G., and A. Adachi. 2000. Requirements for presenilin-dependent cleavage of notch and other transmembrane proteins. Mol Cell. 6:625-36.

1. A method to modulate of modulating phagocytosis of neurons, said method comprising:
modulating the expression of telencephalin expressed on said neurons wherein said modulation is a stimulation of phagocytosis via increased expression of telencephalin, or is a down-regulation of phagocytosis via a decreased expression or a decreased functionality of telencephalin.

2. The method according to claim 1 wherein said stimulation of phagocytosis is via gene transfer of telencephalin.

3. The method according to claim 1 wherein said down-regulation of phagocytosis is by binding a molecule to telencephalin or binding of a molecule to the nucleic acid encoding telencephalin wherein said molecule is chosen from the group consisting of an antibody, an RNAi molecule and an anti-sense molecule.

4. A method of treating an infectious disease of a neuron in a subject, said method comprising:
administering to the subiect an antibody, an RNAi molecule or an anti-sense molecule that binds to telencephalin or to the nucleic acid encoding telencephalin so as to treat the infectious disease.

5. A method of phagocvtosing a complex using telencephalin as an entry site for compounds to enter cells expressing telencephalin, said method comprising.
a) providing an antibody or a ligand for telencephalin,
b) linking said antibody or ligand to a compound resulting in the complex and
c) administrating said complex to cells expressing telencephalin resulting in phagocytosis of said complex.

6. The method according to claim 5 wherein said compound further comprises an antibody against amyloid beta.

7. The method according to claim 5, wherein said cells are neurons.

8. The method according to claim 7 wherein said neurons reside in the telencephalon.

9. The method according to claim 6, wherein said cells are neurons.

10. The method according to claim 9, wherein said neurons reside in the telencephalon.
